(19) United States
(12) Patent Application Publication
(10) Pub. No.: US 2013/0028897 A1
(43) Pub. Date: Jan. 31, 2013

(54) METHODS FOR REDUCING THE LEVEL OF ALLOANTIBODIES IN A SUBJECT

(71) Applicant: Ali Naji, North Wales, PA (US)

(72) Inventor: Ali Naji, North Wales, PA (US)

(21) Appl. No.: 13/266,446

(22) PCT Filed: Apr. 27, 2010
(86) PCT No.: PCT/US10/32599

§ 371 Date: May 15, 2012

Related U.S. Application Data
(60) Provisional application No. 61/173,053, filed on Apr. 27, 2009.

(51) Int. Cl. A61K 39/395; A61P 37/06

(52) U.S. Cl. 424/135.1; 424/142.1; 424/145.1; 424/133.1

(57) ABSTRACT

The invention provides a method for reducing the level of alloantibodies in a sensitized patient awaiting kidney transplantation by administering Belimumab or an antibody that specifically binds a B-lymphocyte stimulator protein (BLyS). The invention further provides a method for reducing the risk of an allograft rejection in an allograft recipient by administering Belimumab or an antibody that specifically binds a BLyS.

### CROSS REFERENCE TO RELATED APPLICATIONS

This application claims priority to U.S. Provisional Patent Application 61/173,053, filed Apr. 27, 2009, which is incorporated by reference herein in its entirety.

### FIELD OF THE INVENTION

The invention relates to compositions and methods for reducing the level of alloantibodies in a subject. Specifically, the invention relates to reducing the level of alloantibodies in a subject by administering to said subject an antibody that specifically binds a B-lymphocyte stimulator protein (BLyS).

### BACKGROUND OF THE INVENTION

Approximately 30% of patients awaiting kidney transplantation have significant detectable alloantibodies which increase their waiting time, or may preclude transplantation. Currently the wait time for a kidney transplant at the University of Pennsylvania Medical Center for patients with Panel Reactive Antibody (PRA) measure in the range of 20%-79% is over 5 years (as compared to patients with low PRA (0%-19%) which is 3-4 years. Antibodies in these patients may be due to prior transplants, pregnancy, or blood transfusions.

Belimumab is a fully human IgG1A antibody that recognizes human BLyS. BLyS™ is a B-lymphocyte stimulator protein which belongs to the tumor necrosis factor (TNF) ligand family. The BLyS gene encodes a type II membrane bound protein expressed on normal monocytes, macrophages and dendritic cells. Granulocyte colony stimulating factor-activated neutrophils also express elevated levels of BLyS mRNA and release increased amounts of biologically active BLyS. In vitro, Belimumab binds soluble, but not membrane bound, BLyS with high affinity and inhibits its biological activity.

Previous clinical studies with Belimumab have been aimed at determining the efficacy, safety, pharmacokinetics, and tolerability of this fully human monoclonal antibody in patients with autoimmune diseases. Pharmacokinetic data revealed that Belimumab has a long half-life (approximately 10 days) and a small volume of distribution relative to extracellular fluid. Belimumab demonstrated a slow clearance which was much less than the glomerular filtration rate, indicating that renal clearance is not a major component of clearance. These pharmacokinetic findings are also similar to those demonstrated in monkey studies with Belimumab.

Belimumab is generally well tolerated with the exception of urticaria which is somewhat higher in Belimumab treated group. The incidence of adverse events, serious adverse events and laboratory abnormalities are generally comparable to placebo.

### SUMMARY OF THE INVENTION

In one embodiment, provided herein is a method for reducing the level of alloantibodies in a subject awaiting kidney transplantation, comprising the step of administering to said subject a therapeutically effective amount of Belimumab, thereby reducing the level of alloantibodies in said subject awaiting kidney transplantation.

In another embodiment, further provided herein is a method for reducing the level of alloantibodies in a subject in need thereof, comprising the step of administering to said subject a therapeutically effective amount of a composition comprising an antibody that specifically binds a B-lymphocyte stimulator protein (BLyS), thereby reducing the level of alloantibodies in said subject in need thereof.

In another embodiment, further provided herein is a method for reducing the risk of an allograft rejection in an allograft recipient, comprising the step of administering to said recipient a therapeutically effective amount of a composition comprising an antibody that specifically binds a B-lymphocyte stimulator protein (BLyS), wherein administering is prior to an organ transplantation, thereby reducing the risk of an allograft rejection in said allograft recipient.

In another embodiment, further provided herein is a method for reducing the wait time for an organ transplant in a patient in need thereof, comprising the step of administering to said patient a therapeutically effective amount of a composition comprising an antibody that specifically binds a B-lymphocyte stimulator protein (BLyS), thereby reducing the wait time for an organ transplant in said patient in need thereof.

In another embodiment, further provided herein is a method of preventing the development of post-transplantation hyperacute rejection in a subject awaiting kidney transplantation, comprising the step of administering to said subject a therapeutically effective amount of a composition comprising an antibody that specifically binds a B-lymphocyte stimulator protein (BLyS), thereby preventing the development of post-transplantation hyperacute rejection in said subject.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

In one embodiment, this invention provides a method for reducing the level of alloantibodies in a subject in need thereof, comprising the step of administering to a subject a composition comprising an antibody that specifically binds a B-lymphocyte stimulator protein (BLyS) or a compound that specifically inhibits BLyS, thereby reducing the level of alloantibodies in a subject in need thereof. In another embodiment, a subject in need thereof is a subject scheduled for an organ transplant. In another embodiment, a subject in need thereof is a subject awaiting for an organ transplant. In another embodiment, a subject in need thereof is a subject that underwent an organ transplant. In another embodiment, a compound that specifically inhibits BLyS differentially inhibits a soluble BLyS and not a membrane bound BLyS. In another embodiment, a compound that specifically inhibits BLyS differentially binds a soluble BLyS and not a membrane bound BLyS.

In another embodiment, a method as described herein prevents the devastating consequences caused by alloantibodies in a patient undergone an organ transplantation. In another embodiment, the devastating consequences caused by alloantibodies in a patient undergone an organ transplantation vary with the organ that is being transplanted. In another embodiment, the devastating consequences caused by alloantibodies are well known to one of average skill in the art. In another embodiment, devastating consequences include graft rejection. In another embodiment, graft rejection due to the presence of alloantibodies can occur immediately after transplantation (hours to few days). In another embodiment, graft rejection due to the presence of alloantibodies can occur weeks to months after transplantation. In another embodiment, graft rejection due to the presence of alloantibodies can occur 0.5-40 years after transplantation.

In another embodiment, provided herein is a method for using Belimumab for normalizing the level of pre-existing alloantibodies in sensitized patients awaiting kidney transplantation. In another embodiment, provided herein is a method for reducing the level of alloantibodies in a subject awaiting kidney transplantation, comprising the step of administering to the subject Belimumab, thereby reducing the level of alloantibodies in a subject awaiting kidney transplantation. In another embodiment, a subject awaiting kidney transplantation is a subject afflicted with an end-stage renal disease. In another embodiment, provided herein is a method for reducing humoral immune responses comprising an alloantibody-mediated responses in a subject afflicted with an end-stage renal disease. In another embodiment, a subject awaiting organ transplantation that is treated by the methods as described herein is a subject having a Panel Reactive Antibody (PRA) measure from 5% to 99%. In another embodiment, a subject awaiting organ transplantation that is treated by the methods as described herein is a subject having a PRA measure from 20% to 79%. In another embodiment, a subject awaiting organ transplantation that is treated by the methods as described herein is a subject having a PRA measure from 2% to 50%. In another embodiment, a subject awaiting organ transplantation that is treated by the methods as described herein is a subject having a PRA measure from 40% to 70%.

In another embodiment, provided herein is a method for reducing alloantibody level in a patient having a PRA measure from 2% to 90% by administering Belimumab, thus permitting subsequent successful kidney transplantation from a cross match compatible donor. In another embodiment, provided herein is a method for converting a cross match incompatible donor to a cross match compatible donor by administering Belimumab to a recipient having high anti-HLA alloantibody level against the donor, thus permitting subsequent successful kidney transplantation. In another embodiment, provided herein is a method for desentisizing a sentisized patient awaiting kidney transplantation, comprising the step of administering Belimumab to the sentisized patient, thus desentisizing a sentisized patient and permitting subsequent successful kidney transplantation. In another embodiment, provided herein is a method for desentisizing a sentisized patient awaiting kidney transplantation, comprising the step of administering Belimumab to the sentisized patient, thus desentisizing a sentisized patient and permitting subsequent successful kidney transplantation from an initially cross match incompatible donor.

In another embodiment, administering Belimumab is intravenously administering Belimumab at a dose of 2-20 mg/kg (body weight). In another embodiment, administering Belimumab is intravenously administering Belimumab at a dose of 2-20 mg/kg (body weight) every 14 days for the initial 2-6 visits and every 28 days for the remaining 20-80 visits. In another embodiment, administering Belimumab is intravenously administering Belimumab at a dose of 2-20 mg/kg (body weight) every 5-20 days for the initial 2-6 visits and every 15-40 days for at least additional 20 visits.

In another embodiment, provided herein is a method for providing sustained reduction in the level of alloantibodies in a subject awaiting kidney transplantation, comprising the step of administering to the subject Belimumab. In another embodiment, provided herein is a method for providing sustained reduction in the level of alloantibodies in a subject that underwent an organ transplantation, comprising the step of administering to the subject Belimumab. In another embodiment, continued treatment with Belimumab stabilizes the level of alloantibodies, in a sensitized patient awaiting kidney transplantation, to a low, normal level which permits transplantation. In another embodiment, continued treatment with Belimumab stabilized, a low, favorible, PAR measure in a sensitized patient awaiting kidney transplantation.

In another embodiment, provided herein is a method for reducing the risk of humoral immune responses such as hyperacute rejection (HAR), acute antibody-mediated rejection, and a more chronic donor-specific alloantibody effect in a subject that underwent a kidney transplantation, comprising the step of administering to the subject, prior to transplantation, a composition comprising an antibody that specifically binds a BLyS. In another embodiment, provided herein is a method for reducing the risk of humoral immune responses such as hyperacute rejection (HAR), acute antibody-mediated rejection, and a more chronic donor-specific alloantibody effect in a subject that underwent a kidney transplantation, comprising the step of administering to the subject, prior to transplantation, Belimumab.

In another embodiment, provided herein is a method for reducing the risk of development of de novo anti-HLA alloantibodies and acute rejection episodes in a subject that underwent an organ transplantation, comprising the step of administering to the subject a composition comprising an antibody that specifically binds a BLyS or a compound that specifically inhibits a BLyS. In another embodiment, provided herein is a method for reducing the risk of development of de novo anti-HLA alloantibodies and acute rejection episodes in a subject that underwent a kidney transplantation, comprising the step of administering to the subject a composition comprising an antibody that specifically binds a BLyS or a compound that specifically inhibits a BLyS. In another embodiment, in order to inhibit development of de novo anti-HLA alloantibodies the composition as described herein is administered after transplantation. In another embodiment, in order to inhibit development of de novo anti-HLA alloantibodies the composition as described herein is administered, periodically, after transplantation. In another embodiment, the allograft is a kidney. In another embodiment, the allograft is a heart.

In another embodiment, the methods as described herein prevent the development of resistance to treatment with conventional immunosuppressive agents. In another embodiment, the methods as described herein normalize the level of preformed alloantibodies and prevent the resistance to treatment with conventional immunosuppression agents. In another embodiment, Belimumab normalizes the level of preformed alloantibodies and prevents the resistance to treatment with conventional immunosuppression agents.

In another embodiment, the methods as described herein induce allograft tolerance. In another embodiment, the methods as described herein protect a patient that underwent an organ transplantation from anti-allograft humoral immune responses. In another embodiment, allograft tolerance is achieved by administering a composition as described herein prior to transplantation. In another embodiment, allograft tolerance is maintained by administering a composition as described herein after transplantation.

In another embodiment, the methods as described herein prevent and/or treat alloantibody-mediated transplant rejection. In another embodiment, the methods as described herein are further combined with traditional methods of alloantibody depletion and immunosuppression.

In another embodiment, provided herein is a method for reducing the risk of an allograft rejection in an allograft recipient, comprising the step of administering to a recipient a composition comprising an antibody that specifically binds a BLyS or a compound that specifically inhibits a BLyS, wherein administering is prior to an organ transplantation, during an organ transplantation, after an organ transplantation, or any combination thereof, thereby reducing the risk of an allograft rejection in an allograft recipient.

In another embodiment, a subject awaiting kidney transplantation is treated with a composition comprising an antibody that specifically binds BLyS for preventing hyperacute rejection after transplantation. In another embodiment, a subject awaiting kidney transplantation is treated with a composition comprising an antibody that specifically binds a BLyS or a compound that specifically inhibits a BLyS, for preventing a hyperacute rejection after transplantation. In another embodiment, a subject awaiting kidney transplantation is treated with a composition comprising an antibody that specifically binds a BLyS or a compound that specifically inhibits a BLyS, for diminishing pre-existing antibodies which induce a hyperacute rejection.

In another embodiment, provided herein is a method of treating a subject afflicted with hyperacute rejection, comprising the step of administering to a subject a composition comprising an antibody that specifically binds a BLyS or a compound that specifically inhibits a BLyS, thereby treating a subject afflicted with hyperacute rejection.

In another embodiment, provided herein is a method of treating a subject afflicted with acute rejection, comprising the step of administering to a subject a composition comprising an antibody that specifically binds a BLyS or a compound that specifically inhibits a BLyS, thereby treating a subject afflicted with acute rejection.

In another embodiment, provided herein is a method for reducing the level of alloantibodies in a sensitized subject, comprising the step of administering to a subject a composition comprising an antibody that specifically binds a BLyS or a compound that specifically inhibits a BLyS. In another embodiment, this invention provides a method for normalizing the level of preformed alloantibodies in candidates awaiting organ transplantation. In another embodiment, this invention provides a method for desensitizing candidates for renal transplantation. In another embodiment, a sensitizing event or events include but are not limited to: pregnancy, blood transfusion, organ transplantation, or any combination thereof.

In another embodiment, an antibody that specifically binds a BLyS inhibits alloantibodies production. In another embodiment, an antibody that specifically binds a BLyS normalizes alloantibody level in a patient having a 20-90% PRA measure. In another embodiment, an antibody that specifically binds a BLyS normalizes alloantibody level in a patient having a 0-20% PRA measure. In another embodiment, an antibody that specifically binds a BLyS normalizes alloantibody level in a patient previously sensitized with an alloantigen. In another embodiment, an antibody that specifically binds a BLyS normalizes anti-HLA alloantibody level in a patient previously sensitized with an alloantigen.

In another embodiment, the invention provides a method for reducing the wait time for an organ transplant, due to high PRA measure, in a patient awaiting organ transplantation. In another embodiment, the method as described herein reduces the wait time by reducing a PRA measure in the patient awaiting organ transplantation.

In another embodiment, this invention reduces the waiting period for transplantation for a patient with a live organ donor by means of minimizing the amount of alloantibodies in the patient. In another embodiment, this invention reduces the waiting period for transplantation for a patient with a live organ donor by means of minimizing the amount of alloantibodies in the donor. In another embodiment, this invention reduces the waiting period for transplantation for a patient with a live kidney donor by means of minimizing the amount of alloantibodies in the patient. In another embodiment, this invention reduces the waiting period for transplantation for a patient with a live kidney donor afflicted with a chronic renal disease by means of minimizing the amount of alloantibodies in the patient. In another embodiment, this invention reduces the waiting period for transplantation for a patient with a live kidney donor having a PRA measure from 10% to 80% by means of minimizing the amount of alloantibodies in the patient.

In another embodiment, provided herein is a method for inhibiting alloimmunity in a subject, comprising the step of administering to a subject a composition comprising an antibody that specifically binds a BLyS or a compound that specifically inhibits a BLyS. In another embodiment, provided herein is a method for inducing tolerance to an alloantigen in a subject, comprising the step of administering to a subject a composition comprising an antibody that specifically binds a BLyS or a compound that specifically inhibits a BLyS.

In another embodiment, provided herein is a method for inhibiting the production of alloantibodies in a recipient of fluids such as blood or plasma, after a transfusion, comprising the step of administering to the recipient a composition comprising an antibody that specifically binds a BLyS or a compound that specifically inhibits a BLyS.

In another embodiment, provided herein is a method for inhibiting the production of alloantibodies in an allograft recipient, comprising the step of administering to the recipient a composition comprising an antibody that specifically binds a BLyS or a compound that specifically inhibits a BLyS. In another embodiment, provided herein is a method for inhibiting the production of alloantibodies in an allograft recipient, comprising the step of administering to the recipient a composition comprising an antibody that specifically binds a BLyS or a compound that specifically inhibits a BLyS before transplantation, during transplantation, and/or after transplantation.

In another embodiment, provided herein is a method for reducing the risk of a hemolytic disease in a subject, comprising the step of administering to the subject a composition comprising an antibody that specifically binds a BLyS or a compound that specifically inhibits a BLyS. In another embodiment, provided herein is a method for reducing the risk of a hemolytic disease of the newborn and/or fetomaternal alloimmune thrombocytopenia, comprising the step of administering to the newborn or fetus a composition comprising an antibody that specifically binds a BLyS or a compound that specifically inhibits a BLyS.

In another embodiment, provided herein is a method for reducing Red cell alloantibodies produced after bone marrow transplantation (BMT), comprising the step of administering to a subject a composition comprising an antibody that specifically binds a BLyS or a compound that specifically inhibits a BLyS. In another embodiment, provided herein is a method for preventing Red cell alloantibodies production after BMT in a subject, comprising the step of administering to a subject a composition comprising an antibody that specifically binds a BLyS or a compound that specifically inhibits a BLyS. In another embodiment, administering the composition as described herein prior to BMT reduces pre-existing Red cell alloantibodies. In another embodiment, administering the composition as described herein during and/or after to BMT inhibits de-novo production of Red cell alloantibodies.

In another embodiment, provided herein is a method for reducing Rh antibodies against pretransplanted red cells in a subject, comprising the step of administering to a subject a composition comprising an antibody that specifically binds a BLyS or a compound that specifically inhibits a BLyS. In another embodiment, provided herein is a method for reducing Rh antibodies against pretransplanted red cells in a subject, following incompatible bone marrow transplantation, comprising the step of administering to a subject a composition comprising an antibody that specifically binds a BLyS or a compound that specifically inhibits a BLyS.

In another embodiment, provided herein is a method for reducing the risk of transmission of an alloantibody from a donor to a recipient during the process of organ and/or cell transplantation, comprising the step of administering to the donor, prior to transplantation, a composition comprising an antibody that specifically binds a BLyS or a compound that specifically inhibits a BLyS. In another embodiment, provided herein is a method for reducing the risk associated with transmission of an alloantibody from a donor to a recipient during the process of organ and/or cell transplantation, comprising the step of administering to the recipient a composition comprising an antibody that specifically binds a BLyS or a compound that specifically inhibits a BLyS, wherein administering is prior to transplantation, during transplantation, and/or after transplantation.

In another embodiment, provided herein is a method for reducing the risk of transmission of an anti-RhD alloantibody from a donor to a recipient during ABO-incompatible BMT, comprising the step of administering to the donor, prior to transplantation, a composition comprising an antibody that specifically binds a BLyS or a compound that specifically inhibits a BLyS. In another embodiment, provided herein is a method for reducing the risk associated with transmission of an anti-RhD alloantibody from a donor to a recipient after ABO-incompatible BMT, comprising the step of administering to the recipient a composition comprising an antibody that specifically binds a BLyS or a compound that specifically inhibits a BLyS, wherein administering is prior to transplantation, during transplantation, and/or after transplantation.

In another embodiment, provided herein is a method for reducing the risk of graft loss due to antibody-mediated rejection in a subject, comprising the step of administering to the subject, a composition comprising an antibody that specifically binds a BLyS or a compound that specifically inhibits a BLyS. In another embodiment, provided herein is a method for reducing the risk of graft loss due to antibody-mediated rejection in a subject, comprising the step of administering to the subject, a composition comprising an antibody that specifically binds a BLyS or a compound that specifically inhibits a BLyS. In another embodiment, administration of a composition comprising an antibody that specifically binds a BLyS or a compound that specifically inhibits a BLyS is preformed prior to transplantation, during transplantation, and/or after transplantation. In another embodiment, a graft is an allograft.

In another embodiment, provided herein is a method for reducing the level of alloantibodies in a patient undergoing non-ABO mismatched allogeneic bone marrow transplantation (BMT), comprising the step of administering to the patient a composition comprising an antibody that specifically binds a BLyS or a compound that specifically inhibits a BLyS. In another embodiment, a patient undergoing non-ABO mismatched allogeneic BMT is at risk of developing alloantibodies against non-ABO red blood cells (RBC) antigens.

In another embodiment, provided herein is a method for reducing the level of alloantibodies in a patient undergoing liver transplantation, comprising the step of administering to the patient a composition comprising an antibody that specifically binds a BLyS or a compound that specifically inhibits a BLyS. In another embodiment, provided herein is a method for reducing the risk of severe delayed hemolysis in a patient undergoing liver transplantation, comprising the step of administering to the patient a composition comprising an antibody that specifically binds a BLyS or a compound that specifically inhibits a BLyS.

In another embodiment, provided herein is a method for diminishing the harmful effects and risks associated with a human organ transplant, wherein the recipient comprises anti-human antibodies in his blood which react with the tissue type of an organ donor, comprising the step of administering to the human organ transplant recipient a composition comprising an antibody that specifically binds a BLyS or a compound that specifically inhibits a BLyS. In another embodiment, provided herein is a method for diminishing the harmful effects and risks faced by a human organ transplant recipient having anti-human antibodies in his blood which react with the tissue type of a kidney donor, comprising the step of administering to the human organ transplant recipient a composition comprising an antibody that specifically binds a BLyS or a compound that specifically inhibits a BLyS. In another embodiment, provided herein is a method for preventing alloantibodies attack and damage of a transplanted kidney. In another embodiment, provided herein is a method for preventing hyperacute rejection in a patient scheduled for kidney transplantation, comprising the step of administering to the patient, prior to transplantation, a composition comprising an antibody that specifically binds a BLyS or a compound that specifically inhibits a BLyS.

In another embodiment, provided herein is a method for enabling kidney transplantation in a patient having a “positive” crossmatch with a donor, comprising the step of administering to the patient, prior to transplantation, a composition comprising an antibody that specifically binds a BLyS or a compound that specifically inhibits a BLyS. In another embodiment, provided herein is a method for enabling kidney transplantation in a patient having a “positive” crossmatch with a donor, comprising the step of administering to the patient, after transplantation, a composition comprising an antibody that specifically binds a BLyS or a compound that specifically inhibits a BLyS. In another embodiment, provided herein is a method for enabling kidney transplantation in a patient having a “positive” crossmatch with a donor, comprising the step of administering to the patient, during transplantation, a composition comprising an antibody that specifically binds a BLyS or a compound that specifically inhibits a BLyS. In another embodiment, provided herein is a method for converting a “positive” crossmatch to a “negative” crossmatch. In another embodiment, provided herein is a method for reducing a “positive” crossmatch.

In another embodiment, provided herein is a method for effective seroconversion. In another embodiment, provided herein is a method for effective seroconversion from a positive alloantibody status to a negative alloantibody status, thereby allowing successful transplantation from initially cross-match incompatible donor to a cross-match compatible donor.

In another embodiment, provided herein is a method for reducing the risk of graft loss in a patient having a Panel Reactive Antibody (PRA) measure of above 0%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure of above 10%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure of above 20%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure of above 30%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure of above 40%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure of above 50%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure of above 60%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure of above 70%, comprising the step of administering to the patient, before transplantation, after transplantation, and/or during transplantation, a composition comprising an antibody that specifically binds a BLyS or a compound that specifically inhibits a BLyS. In another embodiment, the graft is a kidney graft.

In another embodiment, reducing the risk of graft loss is reducing the level of pre-existing alloantibodies, de-novo produced alloantibodies, or their combination.

In another embodiment, provided herein is a method for reducing the risk of graft loss in a patient having a PRA measure from 10% to 90%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure from 50% to 85%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure from 20% to 90%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure from 10% to 50%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure from 15% to 60%, comprising the step of administering to the patient, before transplantation, after transplantation, and/or during transplantation, a composition comprising an antibody that specifically binds a BLyS or a compound that specifically inhibits a BLyS. In another embodiment, provided herein is a method for reducing the risk of graft loss in a patient having a PRA measure from 40% to 80%, comprising the step of administering to the patient, before transplantation, after transplantation, and/or during transplantation, a composition comprising an antibody that specifically binds a BLyS or a compound that specifically inhibits a BLyS.

In another embodiment, provided herein is a method for reducing the wait period for transplantation by at least 20% in a patient having a Panel Reactive Antibody (PRA) measure from 10% to 90%, comprising the step of administering to the patient, before transplantation, a composition comprising an antibody that specifically binds a BLyS or a compound that specifically inhibits a BLyS, thereby reducing the wait period for transplantation by at least 20%. In another embodiment, provided herein is a method for reducing the wait period for transplantation by at least 30% in a patient having a PRA measure from 10% to 90%, comprising the step of administering to the patient, before transplantation, a composition comprising an antibody that specifically binds a BLyS or a compound that specifically inhibits a BLyS, thereby reducing the wait period for transplantation by at least 30%. In another embodiment, provided herein is a method for reducing the wait period for transplantation by at least 40% in a patient having a PRA measure from 10% to 90%, comprising the step of administering to the patient, before transplantation, a composition comprising an antibody that specifically binds a BLyS or a compound that specifically inhibits a BLyS, thereby reducing the wait period for transplantation by at least 40%. In another embodiment, provided herein is a method for reducing the wait period for transplantation by at least 50% in a patient having a PRA measure from 10% to 90%, comprising the step of administering to the patient, before transplantation, a composition comprising an antibody that specifically binds a BLyS or a compound that specifically inhibits a BLyS, thereby reducing the wait period for transplantation by at least 50%. In another embodiment, provided herein is a method for reducing the wait period for transplantation by at least 60% a patient having a PRA measure from 10% to 90%, comprising the step of administering to the patient, before transplantation, a composition comprising an antibody that specifically binds a BLyS or a compound that specifically inhibits a BLyS, thereby reducing the wait period for transplantation by at least 60%.

In another embodiment, provided herein is a method for reducing the wait period for transplantation by at least 10% in a patient having a PRA measure from 20% to 80%, comprising the step of administering to the patient, before transplantation, a composition comprising an antibody that specifically binds a BLyS or a compound that specifically inhibits a BLyS, thereby reducing the wait period for transplantation by at least 10%. In another embodiment, provided herein is a method for reducing the wait period for transplantation by at least 20% in a patient having a PRA measure from 1% to 19%, comprising the step of administering to the patient, before transplantation, a composition comprising an antibody that specifically binds a BLyS or a compound that specifically inhibits a BLyS, thereby reducing the wait period for transplantation by at least 20%. In another embodiment, provided herein is a method for reducing the wait period for transplantation by at least 30% in a patient having a PRA measure from 10% to 90%, comprising the step of administering to the patient, before transplantation, a composition comprising an antibody that specifically binds a BLyS or a compound that specifically inhibits a BLyS, thereby reducing the wait period for transplantation by at least 30%. In another embodiment, provided herein is a method for reducing the wait period for transplantation by at least 40% in a patient having a PRA measure from 10% to 90%, comprising the step of administering to the patient, before transplantation, a composition comprising an antibody that specifically binds a BLyS or a compound that specifically inhibits a BLyS, thereby reducing the wait period for transplantation by at least 40%. In another embodiment, provided herein is a method for reducing the wait period for transplantation by at least 50% in a patient having a PRA measure from 10% to 90%, comprising the step of administering to the patient, before transplantation, a composition comprising an antibody that specifically binds a BLyS or a compound that specifically inhibits a BLyS, thereby reducing the wait period for transplantation by at least 50%.

In another embodiment, the present invention provides a life saving solution for a substantial portion of the patients with the highest PRAs who currently can not undergo transplantation. In another embodiment, the present invention provides a life saving solution for a substantial portion of patients afflicted with a severe kidney disease who also have a high PRA. In another embodiment, this invention provides means for minimizing the amount of alloantibodies in a patient afflicted with an end-stage renal disease having a PRA measure from 10% to 80%. In another embodiment, this invention provides means for minimizing the amount of alloantibodies in a patient afflicted with a chronic renal disease having a PRA measure from 10% to 80%.

In another embodiment, a composition comprising an antibody that specifically binds a BLyS or a compound that specifically inhibits a BLyS reduces PRA measure by at least 10% in a patient having a 20-79% PRA level. In another embodiment, a composition comprising an antibody that specifically binds a BLyS or a compound that specifically inhibits a BLyS reduces PRA measure by at least 20% in a patient having a 20-79% PRA level. In another embodiment, a composition comprising an antibody that specifically binds a BLyS or a compound that specifically inhibits a BLyS reduces PRA measure by at least 30% in a patient having a 20-79% PRA level. In another embodiment, a composition comprising an antibody that specifically binds a BLyS or a compound that specifically inhibits a BLyS reduces PRA measure by at least 40% in a patient having a 20-79% PRA level. In another embodiment, a composition comprising an antibody that specifically binds a BLyS or a compound that specifically inhibits a BLyS reduces PRA measure by at least 50% in a patient having a 20-79% PRA level. In another embodiment, a composition comprising an antibody that specifically binds a BLyS or a compound that specifically inhibits a BLyS reduces PRA measure by at least 60% in a patient having a 20-79% PRA level. In another embodiment, a composition comprising an antibody that specifically binds a BLyS or a compound that specifically inhibits a BLyS reduces PRA measure by at least 70% in a patient having a 20-79% PRA level.

In another embodiment, a composition comprising an antibody that specifically binds a BLyS or a compound that specifically inhibits a BLyS reduces PRA measure by at least 10% in a patient having a 0-19% PRA level. In another embodiment, a composition comprising an antibody that specifically binds a BLyS or a compound that specifically inhibits a BLyS reduces PRA measure by at least 20% in a patient having a 20-79% PRA level. In another embodiment, a composition comprising an antibody that specifically binds a BLyS or a compound that specifically inhibits a BLyS reduces PRA measure by at least 30% in a patient having a 20-79% PRA level.

In another embodiment, a composition comprising an antibody that specifically binds a BLyS or a compound that specifically inhibits a BLyS reduces PRA measure by at least 20% in a patient having a 50-90% PRA level. In another embodiment, a composition comprising an antibody that specifically binds a BLyS or a compound that specifically inhibits a BLyS reduces PRA measure by at least 30% in a patient having a 50-79% PRA level. In another embodiment, a composition comprising an antibody that specifically binds a BLyS or a compound that specifically inhibits a BLyS reduces PRA measure by at least 40% in a patient having a 50-79% PRA level. In another embodiment, a composition comprising an antibody that specifically binds a BLyS or a compound that specifically inhibits a BLyS reduces PRA measure by at least 50% in a patient having a 50-90% PRA level. In another embodiment, a composition comprising an antibody that specifically binds a BLyS or a compound that specifically inhibits a BLyS reduces PRA measure by at least 60% in a patient having a 50-90% PRA level. In another embodiment, a composition comprising an antibody that specifically binds a BLyS or a compound that specifically inhibits a BLyS reduces PRA measure by at least 70% in a patient having a 50-90% PRA level. In another embodiment, a composition comprising an antibody that specifically binds a BLyS or a compound that specifically inhibits a BLyS reduces PRA measure by at least 80% in a patient having a 50-90% PRA level.

In another embodiment, this invention provides a high PRA rescue protocol to assist patients who have a suitable live kidney donor, but who are prevented from receiving the transplant because of a positive crossmatch. In another embodiment, the protocol involves administering to these patients a composition comprising an antibody that specifically binds a BLyS or a compound that specifically inhibits a BLyS. In another embodiment, this invention provides a low PRA maintenance protocol for enhancing kidney transplantation success rates in patients afflicted with an end-stage renal disease, comprising the step of administering to these patients a composition comprising an antibody that specifically binds a BLyS or a compound that specifically inhibits a BLyS. In another embodiment, this invention provides a low PRA maintenance protocol for reducing the risk of an allograft rejection in patients who underwent an organ or cell transplantation, comprising the step of administering to these patients a composition comprising an antibody that specifically binds a BLyS or a compound that specifically inhibits a BLyS. In another embodiment, this invention provides a low PRA maintenance protocol for reducing the risk of hyperacute rejection in patients who underwent an organ or cell transplantation, comprising the step of administering to these patients a composition comprising an antibody that specifically binds a BLyS or a compound that specifically inhibits a BLyS. In another embodiment, the composition is administered prior to transplantation, during transplantation, after transplantation, or any combination thereof.

In another embodiment, reducing the level of alloantibodies comprises inhibiting the production of alloantibodies. In another embodiment, reducing the level of alloantibodies is required in a patient with high PRA levels due to prior transplants, pregnancy, or blood transfusions. In another embodiment, reducing the level of alloantibodies is required in a patient with 20-79% PRA level.

In another embodiment, administration of a composition comprising an antibody that specifically binds a BLyS or a compound that specifically inhibits a BLyS allows patients having a positive cross-match to achieve a negative cross-match after treatment. In another embodiment, administration of a composition comprising a compound that specifically inhibits a BLyS allows patients having a positive cross-match to achieve a negative cross-match after treatment. In another embodiment, a composition comprising a compound that specifically inhibits a BLyS reduces PRA measure by at least 20% in a patient having a 20-90% PRA level. In another embodiment, a composition comprising a compound that specifically inhibits a BLyS reduces PRA measure by at least 20% in a patient having a 50-90% PRA level. In another embodiment, a composition comprising a compound that specifically inhibits a BLyS reduces PRA measure by at least 20% in a patient having a 0-19% PRA level. In another embodiment, the terms “PRA level” and “PRA measure” are used interchangeably.

In another embodiment, the alloantibodies are anti-HLA alloantibodies. In another embodiment, a composition comprising an antibody that specifically binds a BLyS or a compound that specifically inhibits a BLyS is useful for treating an allograft disease. In another embodiment, a composition comprising a compound that inhibits a BLyS or an antibody that specifically binds a BLyS is useful for treating an allograft disease. In another embodiment, a composition comprising a compound that inhibits a BLyS or an antibody that specifically binds a BLyS is useful for treating an allograft disease such as Devil facial tumour disease found in the Tasmanian Devil and canine transmissible venereal tumour which infects dogs.

In another embodiment, methods comprising administering a compound that inhibits a BLyS or an antibody that specifically binds a BLyS are utilzed in patients in need of a kidney transplantation. In another embodiment, patients in need of a kidney transplantation suffer from end-stage renal disease (ESRD), regardless of the primary cause. In another embodiment, methods comprising administering a compound that inhibits a BLyS or an antibody that specifically binds a BLyS are utilzed in patients having a high risk of developing an ESRD. In another embodiment, pathologies or diseases that can induce ESRD include but are not limited to malignant hypertension, infections, diabetes mellitus, glomerulonephritis, polycystic kidney disease, an inborn errors of metabolism, an autoimmune condition, lupus, or Goodpasture's syndrome.

In another embodiment, the transplanted kidney is from a donor that is not genetically similar to the recipient. In another embodiment, the transplanted kidney is from a deceased donor. In another embodiment, the transplanted kidney is from a living donor. In another embodiment, the transplanted kidney is from a living donor who is fit for surgery and has no disease which brings undue risk or likelihood of a poor outcome for either the donor or recipient.

In another embodiment, methods comprising administering a compound that inhibits a BLyS or an antibody that specifically binds a BLyS replace plasmapheresis. In another embodiment, methods comprising administering a compound that inhibits a BLyS or an antibody that specifically binds a BLyS replace IVIG. In another embodiment, methods comprising administering a compound that inhibits a BLyS or an antibody that specifically binds a BLyS are combined with plasmapheresis. In another embodiment, methods comprising administering a compound that inhibits a BLyS or an antibody that specifically binds a BLyS are combined with WIG. In another embodiment, methods comprising administering a compound that inhibits a BLyS or an antibody that specifically binds a BLyS are combined with immunosuppressants.

In another embodiment, kidney transplantation further comprises Kidney-pancreas transplantation. In another embodiment, Kidney-pancreas transplantation is done in patients with diabetes mellitus type I. In another embodiment, Kidney-pancreas transplantation is simultaneous kidney-pancreas transplantation. In another embodiment, Kidney-pancreas transplantation is pancreas after kidney transplantation. In another embodiment, only the pancreas is being transplanted according to the methods of the invention. In another embodiment, transplantation is liver transplantation. In another embodiment, transplantation is cardiac transplantation. In another embodiment, transplantation is multi-organ transplantation.

In another embodiment, methods comprising administering a compound that inhibits a BLyS or an antibody that specifically binds a BLyS are used after transplantation, thus suppressing the production of alloantibodies raised after the introduction of the allograft. In another embodiment, methods comprising administering a compound that inhibits a BLyS or an antibody that specifically binds a BLyS are used after transplantation, thus maintaining stable low PRA measures. In another embodiment, methods comprising administering a compound that inhibits a BLyS or an antibody that specifically binds a BLyS are used in combination with immunosupressants. In another embodiment, methods comprising administering a compound that inhibits a BLyS or an antibody that specifically binds a BLyS are used in combination with tacrolimus. In another embodiment, methods comprising administering a compound that inhibits a BLyS or an antibody that specifically binds a BLyS are used in combination with mycophenolate. In another embodiment, methods comprising administering a compound that inhibits a BLyS or an antibody that specifically binds a BLyS are used in combination with prednisone. In another embodiment, methods comprising administering a compound that inhibits a BLyS or an antibody that specifically binds a BLyS are used in combination with cyclosporine. In another embodiment, methods comprising administering a compound that inhibits a BLyS or an antibody that specifically binds a BLyS are used in combination with rapamycin. In another embodiment, methods comprising administering a compound that inhibits a BLyS or an antibody that specifically binds a BLyS are used in combination with azathioprine.

In another embodiment, methods comprising administering a compound that inhibits a BLyS or an antibody that specifically binds a BLyS enable, sensitized renal dialysis patients lacking a suitable cross-matched negative donor, to successfully pass kidney transplantation. In another embodiment, the methods as described herein overcome the hurdles in finding a successful transplant.

In another embodiment, humoral sensitization and antibody definition are determined by testing patient sera using lymphocytotoxicity assays against at least 60 selected lymphocyte panels from donors with known HLA-A, B type. In another embodiment, lymphocytotoxicity assays detect antibodies that are specific for the products of the HLA-A and B loci.

### Antibodies

In another embodiment, the antibody as described herein is an anti-BLyS antibody. In another embodiment, the antibody as described herein is an anti-human BLyS antibody. In another embodiment, the antibody as described herein is an IgG1λ antibody. In another embodiment, the antibody as described herein is a human IgG1λantibody. In another embodiment, the antibody as described herein is a humanized IgG1λ antibody.

In another embodiment, the antibody as described herein differentially or specifically binds a soluble BLyS. In another embodiment, binding of the antibody as described herein inhibits alloantibodies production. In another embodiment, the actual binding of the antibody as described herein prevents BLyS function. In another embodiment, the actual binding of the antibody to a BLyS as described herein inhibits its biological activity. In another embodiment, in vitro, the antibody as described herein, binds soluble, but not membrane bound, BLyS with high affinity and inhibits its biological activity. In another embodiment, a composition comprising an antibody as described herein is Belimumab. In another embodiment, Belimumab is also known as LymphoStat-B.

In another embodiment, an antibody as described herein is a monoclonal antibody. In another embodiment, an antibody as described herein is a recombinant monoclonal antibody. In another embodiment, one of average skill in the art is familiar with methods of designing and producing BLyS monoclonal antibodies including repertoire cloning or phage display/yeast display.

In another embodiment, an antibody as described herein comprises a binding portion of monoclonal mouse antibody merged with human antibody-producing cell. In another embodiment, an antibody as described herein comprises a binding portion derived from a non-human animal and a non-binding portion derived from human DNA. In another embodiment, an antibody as described herein is a humanized antibody. In another embodiment, an antibody as described is a chimeric antibody. In another embodiment, an antibody as described herein is produced by utilizing a mouse that is genetically engineered to produce more human-like antibodies.

In another embodiment, an antibody as described herein is a single chain variable fragment (scFv) antibody. In another embodiment, an antibody as described herein is a fusion of the variable regions of the heavy and light chains of immunoglobulins, linked together with a short linker. In another embodiment, an antibody as described herein is stripped of the constant regions. In another embodiment, a scFv which specifically binds a BLyS is created by method known to one of average skill in the art. In another embodiment, a scFv which specifically binds a BLyS is created directly from sub-cloned heavy and light chains derived from a hybridoma.

### Pharmaceutical Compositions

In another embodiment, the invention provides a pharmaceutical composition comprising a compound that inhibits a BLyS or an antibody that specifically binds a BLyS and a pharmaceutically acceptable carrier or excipient. In another embodiment, the invention provides a pharmaceutical composition comprising a compound that inhibits a BLyS or an antibody that specifically binds a BLyS as described for inhibiting the production of alloantibodies and/or reducing the level of alloantibodies in a subject awaiting transplantation such as kidney transplantation.

In another embodiment, the invention provides a pharmaceutical composition comprising a combination of active pharmaceutical ingredients comprising a compound that inhibits a BLyS or an antibody that specifically binds a BLyS and an immunosuppressant. In another embodiment, the invention provides a pharmaceutical composition comprising a combination of active pharmaceutical ingredients comprising a compound that inhibits a BLyS or an antibody that specifically binds a BLyS and a renal therapeutic agent.

In another embodiment, the invention provides a pharmaceutical composition comprising Belimumab for inhibiting the production of alloantibodies and/or reducing the level of alloantibodies in a subject awaiting transplantation such as kidney transplantation.

In another embodiment, the invention provides a pharmaceutical composition comprising a combination of active pharmaceutical ingredients comprising Belimumab and an immunosuppressant. In another embodiment, the invention provides a pharmaceutical composition comprising a combination of active pharmaceutical ingredients comprising Belimumab and a renal therapeutic agent.

### Dosages and Routes of Administration

In another embodiment, a composition as described herein is administering to a subject over a period ranging from about one month to about one year. In another embodiment, a subject is a human subject. In another embodiment, a subject is a farm animal. In another embodiment, a subject is a pet. In another embodiment, a subject is a mammal. In another embodiment, a subject is a primate. In another embodiment, a subject is a lab animal. In another embodiment, a subject is a rodent.

In another embodiment, Belimumab is administered at a dose of 2-40 mg/kg. In another embodiment, Belimumab is administered at a dose of 5-30 mg/kg. In another embodiment, Belimumab is administered at a dose of 5-15 mg/kg. In another embodiment, Belimumab is administered at a dose of 8-12 mg/kg. In another embodiment, Belimumab is administered once a week. In another embodiment, Belimumab is administered once every 5-15 days. In another embodiment, Belimumab is administered once every 10-40 days. In another embodiment, Belimumab is administered once every 15-30 days. In another embodiment, Belimumab is administered once every 15-25 days.

In another embodiment, Belimumab is administered at a dose of 2-20 mg/kg on days 0, 14, 28 and every 28 days for the remainder 20-120 weeks prior to transplantation. In another embodiment, Belimumab is administered at a dose of 2-20 mg/kg on days 0, 14, 28 and every 28 days for the remainder 20-120 weeks prior to kidney transplantation. In another embodiment, Belimumab is administered at a dose of 2-20 mg/kg on days 0, 14, 28, 42 and every 28 days for the remainder 40-120 weeks prior to transplantation. In another embodiment, Belimumab is administered at a dose of 2-20 mg/kg on days 0, 14, 28, 42 and every 28 days for the remainder 40-120 weeks prior to kidney transplantation. In another embodiment, Belimumab dosing regimen is determined according to the level of alloantibodies in a patient awaiting for organ transplantation. In another embodiment, Belimumab dosing regimen is determined according to the patient's PRA measure. In another embodiment, dose-schedule is determined according to the patient's PRA measure. In another embodiment, dose-schedule is determined according to the level of alloantibodies in a patient awaiting for organ transplantation.

In another embodiment, a patient awaiting for organ transplantation is destined to pass an organ transplantation. In another embodiment, a patient awaiting for organ transplantation is destined to pass kidney transplantation. In another embodiment, a patient awaiting for kidney transplantation is destined to pass a kidney transplantation.

The methods of this invention can be adapted to the use of the therapeutic compositions comprising a compound that inhibits a BLyS or an antibody that specifically binds a BLyS in admixture with conventional excipients, i.e. pharmaceutically acceptable organic or inorganic carrier substances suitable for parenteral, or enteral (e.g., oral) which do not deleteriously react with the active compounds. Suitable pharmaceutically acceptable carriers include but are not limited to water, salt solutions, alcohols, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. The pharmaceutical preparations are, in some embodiments, sterilized and if desired mixed with auxiliary agents, e.g., lubricants, preservatives, stabilizers, wetting agents, emulsifiers, salts for influencing osmotic pressure, buffers, and the like which do not deleteriously react with the active compounds. They can also be combined where desired with other active agents, e.g., vitamins, steroids, anti-inflammatory compounds, etc., as will be understood by one skilled in the art.

In one embodiment, the route of administration may be parenteral, enteral, or a combination thereof. In another embodiment, the route may be intradermal, subcutaneous, intraperitoneal, intravenous, intra-arterial, intratumoral, parcanceral, transmucosal, intramuscular, intravascular, intraventricular, intracranial, or a combination thereof. In one embodiment, the dosage regimen will be determined by skilled clinicians, based on factors such as exact nature of the condition being treated, the severity of the condition (level od alloantibodies/PRA measure), the age and general physical condition of the patient, etc.

In another embodiment, the compositions include those suitable for oral or parenteral administration, all of which may be used as routes of administration using the materials of the present invention. In another embodiment, the compositions include those suitable for direct injection onto an arterial surface. In another embodiment, the compositions include those suitable for direct injection onto an intraparenchymal injection. The term “parenteral” includes subcutaneous injections, intravenous, intramuscular, intrasternal injection or infusion techniques.

The compositions may conveniently be presented in unit dosage form and may be prepared by any of the methods well known in the art of pharmacy. Methods typically include the step of bringing the active ingredients of the invention into association with a carrier which constitutes one or more accessory ingredients.

Compositions of the present invention suitable for oral administration may be presented as discrete units such as capsules, cachets, tablets or lozenges, each containing a predetermined amount of the compounds of the invention in liposomes or as a suspension in an aqueous liquid or non-aqueous liquid such as a syrup, an elixir, or an emulsion.

Compositions suitable for parenteral administration conveniently comprise a sterile aqueous preparation of the molecule of the invention which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The term “pharmaceutically-acceptable and compatible carrier” as used herein, includes one or more compatible solid or liquid filler diluents or encapsulating substances that are suitable for administration to a human or other animal. In the present invention, the term “carrier” thus denotes an organic or inorganic ingredient, natural or synthetic, with which the compounds of the invention are combined to facilitate application. The term “therapeutically-effective amount” is that amount of the present pharmaceutical composition which produces a desired result or exerts a desired influence on the particular condition being treated. In another embodiment, when the composition is being used as prophylactic additional doses will be administered at periodic intervals after the initial administration. Various concentrations may be used in preparing compositions incorporating the same ingredient to provide for variations in the age of the patient to be treated, the severity of the condition, the duration of the treatment and the mode of administration.

The term “compatible”, as used herein, means that the components of the pharmaceutical compositions are capable of being commingled with a small molecule of the present invention, and with each other, in a manner such that does not substantially impair the desired pharmaceutical efficacy.

Doses of the pharmaceutical compositions of the invention will vary depending on the subject and upon the particular route of administration used. The dose can be delivered at periodic intervals based upon the composition. In another embodiment, compounds might be administered daily, weekly, monthly, or by monthly. Pharmaceutical compositions of the present invention can also be administered to a subject according to a variety of other, well-characterized protocols. For example, using pulsed therapy.

In general, the doses utilized for the above described purposes will vary, but will be in an effective amount to exert the desired anti alloantibodies effect. As used herein, the term “pharmaceutically effective amount” refers to an amount of a compound/antibody which will produce the desired reduction in PRA level in a patient. The doses utilized for any of the above-described purposes will generally be from 1 to about 1000 milligrams per kilogram of body weight (mg/kg), administered one to 10 times per 2 months.

Desired time intervals for delivery of multiple doses of a particular composition can be determined by one of ordinary skill in the art employing no more than routine experimentation.

In another embodiment, the compounds and/or antibodies for use in the present invention may be provided in a single formulation/composition, or in another embodiment, multiple formulations may be used. In one embodiment, the formulations for use in the present invention may be administered simultaneously, or in another embodiment, at different time intervals, which may vary between days, weeks or months.

In one embodiment, the compounds and/or antibodies for use in the invention may be used for acute treatment of temporary conditions, or may be administered chronically, as needed. In one embodiment of the invention, the concentrations of the compounds will depend on various factors, including the nature of the condition to be treated, the condition of the patient, the route of administration and the individual tolerability of the compositions.

In one embodiment, the methods of this invention provide for the administration of the compounds and/or antibodies throughout the life of the subject, or in another embodiment, episodically, or in another embodiment, at prior to organ transplantation. In another embodiment, the patients to whom the compounds and/or antibodies of the invention should be administered are those that are experiencing symptoms of disease or who are at risk of contracting the disease or experiencing a recurrent episode or exacerbation of the disease, or pathological conditions associated with the same.

As used herein, the term “pharmaceutically acceptable carrier” refers to any formulation which is safe, and provides the appropriate delivery for the desired route of administration of an effective amount of at least one compound of the present invention. As such, all of the above-described formulations of the present invention are hereby referred to as “pharmaceutically acceptable carriers.” This term refers to as well the use of buffered formulations wherein the pH is maintained at a particular desired value, ranging from pH 4.0 to pH 9.0, in accordance with the stability of the compounds and route of administration.

For parenteral application, particularly suitable are injectable, sterile solutions, preferably oily or aqueous solutions, as well as suspensions, emulsions, or implants, including suppositories. Ampoules are convenient unit dosages.

The injectable formulations can be also formulated as a dry powder which has to be re-dispersed by addition of the dispersing medium (e.g., water for injection). For suspension formulations, it is essential that they are re-dispersed directly before the application, and that the resulting suspension appears homogenous.

It will be appreciated that the actual preferred amounts of active compound in a specific case will vary according to the specific compound being utilized, the particular compositions formulated, the mode of application, and the particular situs and organism being treated. Dosages for a given host can be determined using conventional considerations, e.g., by customary comparison of the differential activities of the subject compounds and of a known agent, e.g., by means of an appropriate, conventional pharmacological protocol.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

All literature references cited in the present specification are hereby incorporated by reference in their entirety.

The following examples are presented in order to more fully illustrate the preferred embodiments of the invention. They should in no way be construed, however, as limiting the broad scope of the invention.

### EXAMPLES

### Example 1

### Clinical Studies: Phase 1 and 2 Trials in Systemic Lupus Erythrematosus (SLE) Patients

A phase 1 multicenter double-blind, placebo controlled clinical trial of SLE patients treated with belimumab (n=57) or placebo (n=13) was reported (LSBL01). Belimumab was administered intravenously as a single dose or 2 doses 21 days apart at doses of 1, 4 10 and 20 mg/kg. Belimumab was well tolerated at all doses, and there was no apparent correlation between dose and incidence or severity of adverse events. Biological activity was evidenced by a significant reduction in CD20+ cells (p<−0.01 for subjects with baseline values>5% CD20+ cells, pooled across all active cohorts). Patients also experienced reduction in CD138+ cells at some timepoints and a dose related in anti-double stranded deoxyribonucleic acid (anti-dsDNA) and immunoglobulin G (IgG) levels.

Pharmacokinetic data revealed belimumab had a long half-life (approximately 10 days) and a small volume of distribution relative to extracellular fluid. Belimumab demonstrated a slow clearance which was much less than the glomerular filtration rate, indicating that renal clearance is not a major component of clearance. In the Examples below patients have stage 4 or 5 chronic kidney disease.

All patients were evaluated for immunogenicity through 84 days after the last dose of study drug. One of the 57 belimumab treated patients (1.7%), developed an anti-belimumab response that was determined to be neutralizing. The patient received a single 1 mg/kg dose of belimumab and initially developed the response on day 14. This response decreased over time through day 84. An additional patient who received 20 mg/kg in the double dose cohort also developed a potentially positive anti-belimumab response on Day 77, however this response was determined not to be neutralizing and was negative by Day 105. Of note, both patients demonstrated altered belimumab pharmacokinetics. In the study anti-BLyS concentrations are monitored and belimumab pharmacokinetics are performed over time.

A phase 2 multi-center, randomized, double-blind, parallel-group, placebo controlled superiority study of belimumab (LBSL02) was also performed. Patients were administered belimumab (n=336) at doses of 1, 4 or 10 mg/kg or placebo (n=113) on days 0, 14, 28 and every 28 days thereafter for the remainder of the 52 weeks, followed by an optional 24-week extension period during which all patients received belimumab. Patients were to be on stable SLE regimen consisting of any of the following (alone or in combination); prednisone (0-40 mg/day in combination, from 5-40 mg/day alone), antimalarials, nonsteroidal anti-inflammatory drugs, methotrexate, azathioprine, leflunomide, or mycophenolate mofetil. The primary endpoints of percent change in Safety of Estrogen in Lupus National Assessment Trial Systemic Lupus Erythematosus Disease Activity Index disease activity score (known as SELENA SLEDAI) at Week 24 and time to first SLE flare were not met. However, over 70% of belimumab treated patients showed a significant improvement in percent change in disease SELENA SLEDAI activity score at week 52 in patients who were antinuclear antibody (ANA) and/or anti-ds DNA positive (termed autoantibody or seropositive) at baseline. Highlights of other statistically significant data in the overall and seropositive groups included reduction in (and in some cases elimination of anti-dsDNA, in anti-dsDNA positive subjects; and increase in complement C4, in patients with normal or low C4 at baseline. Biological activity was evidenced by significant reductions in CD19+ B cells, CD20+ B cells, naTve B cells, activated B cells and plasmacytoid cells. Memory cells increased within the first month of treatment and slowly decreased to near baseline with continued treatment. Similar results were seen in the 24-week extension.

Belimumab was generally well tolerated with the exception of urticaria which was higher in the belimumab treated group (4% vs 0% placebo p=0.0258). The incidence of adverse events, serious adverse events and laboratory abnormalities were generally comparable to placebo. Overall all infection rates were similar between belimumab and placebo (75.6% vs 72.6%, p=not significant).

### Example 2

### Clinical Studies: Phase 2 Trials in RA Patients

A similar phase 2 trial design to that of SLE patients, has also been performed in RA patients (LBRA01). Patients who failed at least 1 disease modifying anti-rheumatic drug due to toxicity or lack of efficacy, received belimumab (n=214) at 1, 4 or 10 mg/kg or placebo (n=69). Patients were also on a stable RA treatment but concomitant biological response modifiers, such as TNF-a inhibitors were prohibited. Doses were administered intravenously on days 0, 14, 28 and every 29 days thereafter for the remainder of the 24 weeks, followed by an optional 24 week extension with belimumab treatment. At week 24, there was statistically superior American College of Rheumatology 20 responses in the 1 mg/kg group, as well as all active treatment groups. Similar to the findings of the phase 2 SLE study, patients with seropositive RA disease also had improvement in RA scores. Moreover, biological activity was observed as statistically significant reductions in CD19+, CD 20+, naive and activated B-cells and rheumatoid factor. Memory cells increased within the first month of treatment and slowly declined with continued treatment. Similar outcomes were seen in the extension phase. Belimumab was well tolerated at all doses.

### Example 3

### Exploratory Study to Evaluate the Efficacy and Safety of Belimumab for Normalization of Alloantibody Levels in Sensitized Patients Awaiting Kidney Transplantation

Approximately 30% of patients awaiting kidney transplantation have significant detectable alloantibodies which increase their waiting time, or may preclude transplantation leading to a significantly prolonged wait time and mortality. The waiting time for patients with panel reactive antibody (PRA) in the range of 20%-79% is over 5 years as compared to patients with low PRA (0%-19%) which is 3-4 years. Patients with a PRA greater than 80% are likely to get allocated extra points to enhance the chances of transplantation. To assess the efficacy of belimumab on the anti-HLA antibody level, the target population are patients with a PRA of 20-79%. Antibodies in these patients may be due to prior transplants, pregnancy, or blood transfusions. This study is undertaken to assess the efficacy and safety of using belimumab to normalize alloantibody levels in sensitized patients awaiting kidney transplantation. Modulating alloantibody levels allow these patients to have a negative cross-match and result in eventual successful kidney transplantation.

To study belimumab in sensitized patients listed for kidney transplant, twenty pre-transplant patients fulfilling inclusion/exclusion criteria are enrolled into the study as described below. This study assesses the efficacy and safety of using Belimumab administered intravenously on days 0, 14, 28 and every 28 days for the remainder 52 weeks to normalize alloantibody levels in sensitized patients awaiting kidney transplantation.

Effectiveness is defined as seroconversion to a negative alloantibody status thereby allowing successful transplantation from a cross-match compatible donor.

### Population

Twenty highly sensitized adult patients listed for kidney transplant are being recruited into the study pre-transplant. Each patient must meet the inclusion/exclusion criteria and sign an informed consent form prior to study entry.

Inclusion criteria includes: Male or female patients aged 18-75 years; Patients denied a kidney transplant because of a prior positive crossmatch; Patients awaiting a first or second kidney transplant from a living or deceased donor; Patients who have given written informed consent to participate in all aspects of the study; Pre-sensitized patients defined by Luminex antibody assays and whose PRA can vary between 15% and 79%; Patients with no potential living donors should have accumulated an excess of at least 12 months over and above the median waiting time for their blood group in the OPO.

Exclusion criteria includes: Patients with known hypersensitivity to Belimumab or who have received biologies, within the last 90 days; Patients receiving intravenous immunoglobulin, cyclophosphamide, mycophenolate mofetil, or azathioprine within the last 90 days; Patients with a history of anaphylaxis to parenteral administration of contrast agents, foreign proteins, or monoclonal antibodies; Patients with multi-organ transplant; Patients who have received any investigational immunosuppressive drug within 1 month of inclusion into this study or if use of such a product is anticipated; Patients who have received any live vaccine within 30 days of study entry; Patients receiving intravenous immunoglobulin, cyclophosphamide, mycophenolate mofetil, or azathioprine within the last 90 days; Patients with a known malignancy or history of malignancy other than excised basal or squamous cell carcinoma of the skin; HBV, HCV or HIV-positive patients; Patients with evidence of severe liver disease, including abnormal liver profile (aspartate aminotransferase (AST), alanine aminotransferases (ALT) or total bilirubin>3 times upper limit of normal at screening); Patients with current severe infection; Patients with any surgical or medical condition, which in the opinion of the investigator precludes enrollment in this trial; Patients who live far from the transplant center and are unable to comply with all study visits; Female patients who are pregnant, lactating or of child bearing potential and not practicing an approved method of birth control. A female subject is eligible to enter the study if she is: Not pregnant or nursing; Of non-childbearing potential (ie, women who had a hysterectomy, are postmenopausal which is defined as 1 year without menses, have both ovaries surgically removed or have current documented tubal ligation); or of non-childbearing potential (i.e., women with functional ovaries and no documented impairment of oviductal or uterine function that would cause sterility). This category includes women with oligomenorrhoea (even severe), women who are perimenopausal or have just begun to menstruate. These women must have a negative serum pregnancy test at screening, and agree to 1 of the following: Complete abstinence from intercourse from 2 weeks prior to administration of the 1st dose of study agent until 8 weeks after the last dose of study agent; or Consistent and correct use of 1 of the following acceptable methods of birth control for 1 month prior to the start of the study agent and 8 weeks after the last dose of study agent: Implants of levonorgestrel; Injectable progesterone; Any intrauterine device (IUD) with a documented failure rate of less than 1% per year; Oral contraceptives (either combined or progesterone only); Double barrier method: Condom, cervical cap or diaphragm with spermicidal agent; Transdermal contraceptive patch; Male partner who is sterile prior to the female subject's entry into the study and is the sole sexual partner for the female subject.

A male subject is eligible to enter the study if he agrees to use effective contraception throughout the study and for 3 months after the last dose of study agent.

### Study Design

All patients receive Belimumab at a dose of 10 mg/kg on days 0, 14, 28 and every 28 days for the remainder 52 weeks.

Patients who have previously received IVIG or patients with a history of allergies (allergic responses to food, drugs, insects, or a history of urticaria), are premedicated 1 hour prior infusion with 25 mg of diphenhydramine or equivalent administered orally and 650 mg of acetaminophen administered orally.

B lymphocyte subsets through flow cytometry, and BLyS levels are evaluated pre-treatment and once every two months after belimumab administration. The effect of belimumab on serial flow crossmatch, PRA assays and anti-HLA antibody specificity are also evaluated pre-treatment and once every two months. For belimumab pharmacokinetic assessment, a sparse sampling scheme is used in this study and all subjects are sampled for serum belimumab levels.

Clinical evaluations and safety laboratory values are monitored at predetermined time points. Adverse and serious adverse events, infectious episodes and newly diagnosed malignancies are recorded during the course of the study.

Belimumab is supplied by Human Genome Sciences, as a lyophilized or liquid formulation in sterile single-use vials. Belimumab is filled into Type 1 glass vials, sealed with a latex free rubber stopper and a flip-off seal, and stored at 2 to 8° C. Each is of 80 mg/mL strength after reconstitution. The vial should not be shaken and frothing is to be avoided.

The reconstituted study agent is diluted in 250 mL normal saline for IV infusion. An amount of normal saline, equal to the calculated amount of product to be added, should be removed from the infusion bag prior to adding the product. After adding the reconstituted product, gently invert the bag to mix the solution. The prepared study agent is infused over 1 hour. This rate of infusion is slowed or interrupted if the subject appears to develop any signs of adverse reaction or other infusion-associated symptoms. Investigators who administer an exogenous protein such as belimumab must be well equipped to assess and manage anaphylactic reactions that may occur. Investigators are encouraged to follow their standard practices to manage any untoward infusion reactions noted during the infusion period. The study drug once reconstituted must be used within eight hours and should be stored at 2 to 8° C. or room temperature. If not used within 8 hours, the reconstituted solution should be discarded.

### Medication

Belimumab is used only for patients participating in the study. Following the infusion, patients are observed for the duration of one hour. All Belimumab doses injected are recorded on the Dosage Administration Record CRF. Patients who are unable to tolerate the protocol-specified dosing schedule are withdrawn from the study.

A sparse sampling scheme is used in this study and all subjects are sampled for serum Belimumab levels. A blood sample for pharmacokinetic analysis is drawn on Days 0 (prior to dosing), 14 (0-4 h after end of infusion), 56 (prior to dosing), 168 (0-4 h after end of infusion), 364 (prior to dosing), at any time (3-7 days post dose) and at unscheduled visit.

Study clinic visit 1 occurs prior to Belimumab infusion, for enrollment and informed consents and screening. Visits 2-15 occur on Days 0, 14, 28 and every 28 days thereafter at Months 2, 3, 4, 5, 6, 7, 8, 9, 10, 11, and 12 for ongoing Belimumab infusions, respectively. Additional follow-up visits occur according to the center's standard of care, during treatment and after treatment has concluded. Patients are being encouraged to return for all evaluations, as scheduled. However, if necessary, patients are seen ±7 days of the scheduled date for visits 2 to 15. The evaluation and visit schedule is summarized in Table 7-1.

At visit 1, 5, 7, 9, 11, 13 and 15 patients undergo a complete physical exam and the relevant medical history is documented in the source document. Patients also undergo measurement of laboratory parameters such as: chemistries, complete blood count, panel reactive antibodies, Anti-BLyS antibodies, anti-HLA specificity antibodies, B lymphocyte subsets, as well as chemistries including liver function tests at visits 4, and 6. At visits 2 to 15 when Belimumab is infused, are documented. Pregnancy tests are performed every 4 weeks during treatment and at the 8 week follow-up visit. Prior and concomitant medications are recorded at each visit.

Venous blood samples are collected on Days 0 (prior to dosing), 14 (0-4 h after end of infusion), 56 (prior to dosing), 168 (0-4 h after end of infusion), 364 (prior to dosing), at any time (3-7 days post dose) and at unscheduled visit.

The following background information is collected at baseline: Recipient data: recipient age, gender, ethnicity, cause of end stage renal disease, prior transplants, rejection episode type if prior transplant, history of blood transfusions, pregnancy.

Patients awaiting transplant receive any non-immunosuppressive prophylactic or therapeutic concomitant medication while in the study as clinically indicated. All concomitant medication given at entry and continuing during the study, including electrolyte supplements, and any new therapies administered are recorded in the CRF giving the reason for the therapy.

No additional investigational drugs other than the study medication (belimumabare administered during the study period pre-transplant.

Patients may not receive intravenous immunoglobulin, mycophenolate mofetil, cyclophosphamide, and azathioprine or any biologies within 90 days of study entry and during the pre-transplant study period. No patient receives a live vaccine during study course or for at least three months therafter.

The investigator instructs the patient to notify the study site about any new medications he/she takes after the start of the study drug.

Efficacy and Safety Assessments include: Incidence of negative cross-match, ability to receive a compatible kidney transplant, transplant success or failure (defined as ability to retain a functioning allograft at 6 and 12 months post-transplant) following the desensitization protocol, serial flow crossmatches, PRA assays and anti-HLA antibody specificity, B lymphocyte subsets through flow cytometry and BLyS levels, Evaluation of Belimumab pharmacokinetics, Change in vital signs (such as systolic, diastolic and mean blood pressure) and lab parameters (such as complete blood count, chemistries, liver function tests), Calculated GFR (MDRD and Nankivell), Rate, type and severity of clinical biopsy proven acute rejection (as determined by Banff 97 grading), Rate of adverse events (AES) including severe infusion reactions.

### Data Analysis

Descriptive and summary statistics are being provided for all measurements and data. In the pilot study 20 patients are chosen. For pharmacokinetic assessment, serum Belimumab concentration data is analyzed using population approach. Serum concentrations at each time point is presented using appropriate graphic and tabular summaries.

This study is undertaken to evaluate an alternative, effective treatment strategy for desensitization of sensitized patients awaiting kidney transplantation. It establishes the pharmacokinetic parameters of Belimumab after multiple dosing and optimal dose to achieve a negative alloantibody status in sensitized patients. All clinical observations are descriptive.

A severity assessment of Life-threatening is not necessarily the same as the seriousness criterion of Life-threatening. The former means that the event is a potential threat to life. The latter means that the event is an immediate threat to life.

### Study Drug Discontinuation

Belimumab is discontinued if a patient develops a significant adverse event with suspected relationship to Belimumab.

Patients who undergo kidney transplantation receive immunosuppressive medication (induction with poiyclonal anti-T cell antibody (Thymoglobulin); and maintenance with micophenolate mofetil (Cellcept), calcineurin inhibitor (Prograf), steroids) as clinically indicated, but no longer receive continued treatment with belimumab.

A Study Drug Discontinuation form is completed, giving the date and reason for stopping the study drug.

Patients who discontinue study drug before the last dose of belimumab are scheduled for a visit 4 weeks after the last dose of belimumab, at which time all of the assessments listed for the final visit are performed and entered in the End of study CRF. In case they don't return for this visit, they are contacted for safety evaluations 25-30 days following the last dose of study drug. Information on why the treatment was discontinued along with 8 week follow up data is requested for all patients.

### Premature Patient Withdrawal

Patients voluntarily withdraw or are dropped from the study at the discretion of the investigator at any time. Patients must be withdrawn from the study if any of the following occur: pregnancy, subject withdrew consent, grade 3 abnormal liver function tests, lost to follow-up, malignancy, life-threatening infection, death.

If such withdrawal occurs, or if the patient fails to return for visits, the investigator determines the primary reason for a patient's premature withdrawal from the study and record this information on the Study Completion CRF.

Patients are withdrawn from the study prematurely for one of the following reasons: adverse event(s), abnormal laboratory value(s), abnormal test procedure result(s), condition no longer requires treatment, repeated protocol violations, administrative problems.

If a patient discontinues study drug and is prematurely withdrawn from the study, the reason given should correspond to one of the above listed possibilities.

For patients who are lost to follow-up, the investigator should show “due diligence” by documenting in the source documents steps taken to contact the patient, e.g., dates of telephone calls, registered letters, etc.

Patients who are prematurely withdrawn are not replaced. Withdrawn patients are not eligible for extended treatment with belimumab.

### Example 4

### Visit Schedule and Assessments

### Schedule of Assessments

Table 1.1 and 1.2 lists all of the assessments pre and post-transplant and indicates with an “X” the visits when they are performed. Patients who undergo transplantation no loner continue belimumab treatment, but undergo 12 months follow-up. All data obtained from these assessments are supported in the patient's source documentation. Assessments that generate data for database entry and which are recorded on CRFs are listed using the CRF name. Assessments that are transferred to the database electronically (e.g., laboratory data) are listed by test name.

Patients who receive transplantation during the study, and therefore no longer continue with belimumab treatment undergo the same schedule of assessments in addition to their post-transplant standard of care.

TABLE 1.1
Schedule of assessments pre-transplant

| | [symbol] | | | | | | | | | | | | | | |
|---|---|---|---|---|---|---|---|---|---|---|---|---|---|---|---|
| | ( [symbol] | | | | | | | | | | | | | | |
| | or | | Day | Day | | | | | | | | | | | |
| | [symbol] ) | Day 0 | 14 | [symbol] | M2 | M3 | M4 | M5 | M6 | M7 | M8 | M9 | M10 | [symbol] | [symbol] |
| Visit | 1 | 2 | 3 | 4 | 5 | 6 | 7 | 8 | 9 | 10 | 11 | 12 | 13 | 14 | 15 |
| [symbol] | X | | | | | | | | | | | | | | |
| [symbol] | | | | | | | | | | | | | | | |
| [symbol] | X | | | | | | | | | | | | | | |
| [symbol] | | | | | | | | | | | | | | | |
| Medical history | X | | | | | | | | | | | | | | |
| Demographic | X | | | | | | | | | | | | | | |
| Information | | | | | | | | | | | | | | | |
| [symbol] | | | | | | | | | | | | | | | |
| Complete physical | X | | | | | | | | x | | | | | | x |
| exam | | | | | | | | | | | | | | | |
| Vital signs | x | x | X | X | X | X | X | X | X | X | X | X | X | X | x |
| Temperature, HR, | | | | | | | | | | | | | | | |
| BP, Weight in kg) | | | | | | | | | | | | | | | |
| Height in meters | X | | | | | | | | | | | | | | |
| Complete Blood | X | | | | x | | x | | x | | x | | x | | x |
| Chemistry (WBC w/ | | | | | | | | | | | | | | | |
| diff, PLT, Hgb, HCT) | | | | | | | | | | | | | | | |
| Metabolic Panel | x | | | x | x | x | x | | x | | x | | x | | x |
| (BUN, creatinine, Na+, | | | | | | | | | | | | | | | |
| K+, HCO3−, fasting | | | | | | | | | | | | | | | |
| glucose, Ca, PO4, Mg, | | | | | | | | | | | | | | | |
| PTH, Total Bilirubin, | | | | | | | | | | | | | | | |
| GGT, ALT, AST, ALKP, | | | | | | | | | | | | | | | |
| uric acid) | | | | | | | | | | | | | | | |
| Pregnancy test | X | x | x | x | x | x | x | x | x | x | x | x | x | x | x |
| [symbol] | | | | | | | | | | | | | | | |
| [symbol] | | | | | | | | | | | | | | | |
| Serology: HBV, HCV, | X | | | | | | | | | | | | | | |
| HIV | | | | | | | | | | | | | | | |
| [symbol] | X | | | | x | | x | | x | | x | | x | | x |
| [symbol] | | | | | | | | | | | | | | | |
| Anti-HLA specificity | x | | | | x | | x | | x | | x | | x | | X |
| [symbol] | x | | | | x | | x | | x | | x | | x | | X |
| B lymphocyte | x | | | | x | | x | | x | | x | | x | | X |
| subsets | | | | | | | | | | | | | | | |
| [symbol] | | x | x | x | x | x | x | x | x | x | x | x | x | x | X |
| [symbol] | | | | | | | | | | | | | | | |
| Pharmacokinetic | | x | X [symbol] | | x | | | X | | | | X (Day | x | x | x |
| evaluations | | [symbol] | [symbol] | | (day | | | (Day | | | | [symbol] | | | |
| | | [symbol] | [symbol] | | [symbol] | | | [symbol] | | | | [symbol] | | | |
| | | | | | [symbol] | | | [symbol] | | | | [symbol] ) | | | |
| | | | | | [symbol] ) | | | [symbol] ) | | | | | | | |
| Adverse and Serious | | x | x | x | x | x | x | x | x | x | x | x | x | x | X |
| Adverse Events | | | | | | | | | | | | | | | |
| Prior and | x | x | x | x | x | x | x | x | x | x | x | x | x | x | x |
| concomitant | | | | | | | | | | | | | | | |
| medications | | | | | | | | | | | | | | | |

1. Pregnancy test will also be performed at the [symbol] week follow-up visit following completion of drug
2. Blood for pharmacokinetics will also be drawn at any time (3-7 days post dose) and at unscheduled visits) [symbol]
indicates data missing or illegible when filed

TABLE 1.2
Schedule of assessments post-transplant

| | | | | | | Wk |
|---|---|---|---|---|---|---|
| | | | | Wk | Wk | 52 |
| | | | Wk 4 | 12 | 24 | Mo |
| | Day 0 | Wk 1 | Mo 1 | Mo 3 | Mo 6 | 12 |
| Visit | 1 | 2 | 4 | 5 | 6 | 7 |
| Donor CMV serology | X | | | | | |
| Donor and Recipient | x | | | | | |
| HCV, HIV and HBV | | | | | | |
| serology | | | | | | |
| Donor demographics | X | | | | | |
| (cause of death, ECD | | | | | | |
| or not, cold ischemia | | | | | | |
| time) | | | | | | |
| PRA | x | | | | | |
| HLA Mismatch | x | | | | | |
| Complete physical | x | x | x | x | x | X |
| exam | | | | | | |
| Vital signs | x | x | x | x | x | X |
| (Temperature, HR, | | | | | | |
| BP, Weight in kg) | | | | | | |
| Complete Blood | x | x | x | x | x | X |
| Chemistry (WBC w/ | | | | | | |
| diff, PLT, Hgb, HCT) | | | | | | |
| Metabolic Panel | x | x | x | x | x | X |
| (BUN, creatinine, Na+, | | | | | | |
| K+, HCO3−, fasting | | | | | | |
| glucose, Ca, PO4, Mg, | | | | | | |
| PTH, Total Bilirubin, | | | | | | |
| GGT, ALT, AST, ALKP, | | | | | | |
| uric acid) | | | | | | |
| Tacrolimus trough | | x | x | x | x | X |
| level | | | | | | |
| MPA trough level | | x | x | x | x | X |
| Anti-HLA specificity | x | | x | x | x | X |
| BK Virus Blood | | | x | x | x | X |
| Quant PCR | | | | | | |
| Prior and | x | x | x | x | x | X |
| concomitant | | | | | | |
| medications | | | | | | |
| Opportunistic | X | x | x | x | x | X |
| infections | | | | | | |
| Rejection episodes | X | x | x | x | x | X |
| Chronic Allograft | X | x | x | x | x | X |
| Nephropathy | | | | | | |
| Estimated GFR | | x | | | x | X |
| BLyS Level | x | | x | x | x | |
| B Lymphocyte | x | | x | x | x | |
| Subsets | | | | | | |

### Information to be Collected on Consented but not Randomized Patients

The following information is collected on patients who are screened and consented but deemed ineligible to receive therapy in the study:assigned patient number, date of birth, gender, ethnicity, peak and current panel reactive antibody, HLA sub-typing, reason not eligible for study. The above information is documented in the screening log.

### Patient Demographics/Other Baseline Characteristics

The following patient information is collected on consented eligible patients: unique study number, date of birth, recipient ethnicity, recipient gender, height and weight, HLA typing, PRA (peak and current), cause of end stage renal disease, medical history (including and not limited to: history of previous transplants, graft rejection reasons in prior transplant recipients, end stage renal disease, history of blood transfusions, pregnancies), pre-transplant serologies such as HCV, HBV, HIV.

### Primary Efficacy Variables

This descriptive study establishes the efficacy and safety of belimumab as is relates to normalizing alloantibody in highly sensitized patients awaiting kidney transplant.

### Secondary Efficacy Variables

To determine whether reduction in alloantibody levels by belimumab therapy permits subsequent successful kidney transplantation from a cross-match compatible donor.

### Safety

The assessment of safety are based mainly on the frequency of adverse and serious adverse events and on the number of laboratory values that fall outside of pre-determined ranges. Other safety data (e.g. vital signs, physical findings, special tests) is considered as appropriate.

### Adverse Events

The occurrence of adverse events are sought by non-directive questioning of the patient at each visit during the study. Adverse events are also detected when they are volunteered by the patient during or between visits or through physical examination, laboratory test, or other assessments.

Information about known adverse events experienced by subjects administered the investigational drug is found in the Investigator Brochure or is communicated between IB updates in the form of Investigator Notifications. This information is included in the patient informed consent and is discussed with the patient during the study as needed.

### Unexpected Adverse Event

An unexpected adverse event is an adverse event, the nature and severity of which is not consistent with the applicable product information (e.g. Investigator Brochure for an unapproved study agent or package insert/summary of product characteristics for an approved product).

### Expected Adverse Event

An expected adverse event means that the event has previously been observed with the study agent and it is identified or described in the applicable product information. It does not mean that the event is expected with the underlying disease(s) or concomitant medications.

### Laboratory Adverse Event

A laboratory abnormality is reported as an adverse event if it is associated with an intervention. Intervention includes, but is not limited to, discontinuation of treatment, dose reduction/delay, or concomitant therapy. In addition, any medically important laboratory abnormality is reported as an adverse event at the discretion of the investigator. This includes laboratory abnormalities for which there is no intervention but the abnormal value(s) suggests a disease or organ toxicity. If clinical sequelae are associated with a laboratory abnormality, the diagnosis or medical condition should be reported (eg, renal failure, hematuria) not the laboratory abnormality (eg, elevated creatinine, urine RBC increased).

Laboratory tests are graded according to the Adverse Event Severity Grading Tables. If a particular lab test is not listed, the lab test should be graded mild, moderate, severe or life-threatening.

### Serious Adverse Event

AnSAE is defined as an adverse event resulting in any of the following outcomes:death, life-threatening (ie an immediate threat to life), inpatient hospitalization, prolongation of an existing hospitalization, persistent or significant disability/incapacity, congenital anomaly/birth defect, is medically important.

An inpatient hospitalization is defined as an admission for any length of time. A hospitalization for administration of study agent, for routine or planned clinical procedures, or for “social” reasons (not the result of any adverse change in the subject's condition) is not considered an adverse event and should not be reported as a serious adverse event. If the subject experiences any adverse change in condition during hospitalization, the condition is reported as an adverse event or serious adverse event according to the above definitions.

Medical and scientific judgment is exercised in deciding whether expedited reporting is appropriate in other situations, such as important medical events that are not be immediately life-threatening or result in death or result in hospitalization but jeopardize the patient or require intervention to prevent one of the other outcomes listed above. These are also usually considered serious. (ICH guidelines, March 1995)

Unlike routine safety assessments, SAEs are monitored continuously and have special reporting requirements.

### Investigator Evaluation of Adverse Events

The investigator evaluates all adverse events with respect to seriousness (criteria listed above), severity (intensity or grade) and causality (relationship to study agent). The criteria for serious are listed above. The severity of an AE is evaluated according to the Adverse Event Severity Grading Tables. The following severity classifications will be used: grade 1—mild-causing no limitation of usual activities, grade 2—moderate-causing some limitation of usual activities, grade 3−—severe-causing inability to carry out usual activities, grade 4—life-threatening—potentially life-threatening or disabling; significant medical intervention is required.

### Causality

Causality is assessed according to the following criteria: definitely related, where there is a reasonable temporal relationship to study agent administration, follows a known response pattern (ie, drug is known to cause this AE), and there is no alternative etiology; probably related, where there is reasonable temporal relationship, follows a suspected response pattern (ie, based on similar drugs), where there is no evidence for a more likely alternative etiology; possibly related, where there is reasonable temporal relationship, and little evidence for a more likely alternative etiology; probably not related, where there does not have a reasonable temporal relationship or where there is good evidence for a more likely alternative etiology; not related, where there does not have a temporal relationship or definitely due to alternative etiology.

The causality assessment is made by the investigator based on information available at the time that the SAE worksheet is completed. The initial causality assessment is revised as new information becomes available.

All adverse events are treated appropriately. Such treatment includes changes in study drug treatment including possible interruption or discontinuation, starting or stopping concomitant treatments, changes in the frequency or nature of assessments, hospitalization, or any other medically required intervention. Once an adverse event is detected, it is followed until its resolution, and assessment is made at each visit (or more frequently, if necessary) of any changes in severity, the suspected relationship to the study drug, the interventions required to treat it, and the outcome.

### Follow-up of Adverse Events

Adverse events that occur from the start of study agent administration through 8 weeks after the date of last administration of study agent are followed until final outcome is known or until the end of the 8-week study follow-up period. Adverse events that have not resolved at the end of the 8-week study follow-up visit are recorded on the adverse event case report form (AE eCRF) as ONGOING.

For subjects participating in the continuation protocol, AEs and SAEs are followed and documented for this study until the subject receives the 1st dose of study agent in the continuation protocol (generally until the Exit visit 4 weeks after the last dose of study agent on this protocol).

For subjects who are not participating in the continuation protocol, new SAEs and SAEs that have not resolved by the 8-week follow-up visit are followed until final outcome is known. If it is not possible to obtain a final outcome for a particular SAE (eg, the subject is lost to follow-up), then the reason a final outcome could not be obtained must be documented by the investigator. For subjects entering the continuation protocol, new SAEs and SAEs that have not resolved are followed under the new protocol.

### Post Study Serious Adverse Events

SAEs that occur after the follow-up period (ie, 8 weeks after the last dose of the study agent) that are assessed by the investigator to be possibly, probably, or definitely related to study agent are reported to the Drug Safety designee on the SAE worksheet as described in SAE section. Post study SAEs are not documented on the AE eCRF.

### Graft Biopsies/Acute Rejection Episodes

In case of clinically suspected acute rejection a percutaneous graft biopsy are obtained. Rejection episodes and chronic allograft nephropathy will be graded by Banff 97 criteria. C4d staining and donor specific antibodies are performed on patients who have biopsies suspicious for antibody mediated rejection. The result is recorded in the CRF as a biopsy proven event with or without treatment.

### Infections

All suspected infections are fully investigated and are documented on the Infections CRF page with details of the diagnosis, name of microorganism (if known), sample site, event causality, relationship to study medication, severity, duration of symptoms (if present), treatment, outcome, and whether or not the infection meets the definition of “serious”.

The criteria for a “serious” infection conform to those for a “serious” adverse event. The microorganism is documented as unknown, suspected, not cultured, or proven. Any anti-infective therapy administered is recorded. Serious infections are reported according to the procedures listed for reporting SAEs.

### Physical Examination

A full physical examination is done at specified visits. Depending on reported symptoms, correlated physical findings are noted in the source document and CRF.

### Vital Signs

At all visits, physical examination results of temperature, heart rate, sitting blood pressure and weight are noted in the source document and CRF.

### Laboratory Evaluations

All standard laboratory samples (see Schedule of Assessments) for clinical evaluation is measured at the local hospital laboratory of the transplant centers involved. Blood samples are taken prior to the administration of belimumab (where appropriate) at screening and then at every scheduled visit thereafter.

The following parameters will be measured at visits 1, 5, 7, 9, 11, 13 and 15 and visits 5, 7, 9, 11, 13 and 15 in the extension to week 104: Panel reactive antibodies and HLA specific antibodies using a combination of Luminex bead assays which include screening with mixed beads and determination of specificity with Luminex specificity and/or Luminex Single Antigen assays. Antibody titers and fluorescence intensity are monitored to assess the effect of the belimumab on preexisting anti-HLA antibody response; BLyS levels and B lymphocyte subsets after belimumab therapy; hematology, including, hemoglobin, WBC and differential, and platelet count; metabolic panel, including sodium, potassium, bicarbonate, calcium, phosphate, magnesium, BUN, creatinine, fasting glucose, PTH, total bilirubin, GGT, ALT, AST, alkaline phosphatase, uric acid; for female patients of child bearing age, a pregnancy test is obtained on all days belimumab is administered prior to drug administration; belimumab pharmacokinetics is performed on days 0, 14, 56, 168, 364 and at any time (3-7 days post dose) and at unscheduled visits.

The following parameters are measured at visit 1 only: Serologies for HBV, HCV, HIV, EBV and CMV is reported prior to the kidney transplant.

### Tolerability

The presence or absence of signs and symptoms related to belimumab injection are noted in source documents and CRF.

### Pharmacokinetics

Venous blood samples are collected on Days 0 (prior to dosing), 14 (0-4 h after end of infusion), 56 (prior to dosing), 168 (0-4 h after end of infusion), 364 (prior to dosing), at any time (3-7 days post dose) and at unscheduled visit.

At each sampling time point, a minimum of 2 mL blood is withdrawn into a collection tube without anticoagulant. After allowing the blood to clot at room temperature, serum is harvested by centrifugation, a minimum of 1 ml transferred to a polypropylene tube, and frozen at −18° C. to −70° C.

Complete pharmacokinetic assessments are performed at to obtain consistent data with minimal variability.

### Example 5

### Safety Monitoring

### Serious Adverse Event Reporting

All adverse events (AEs) that are identified from the start of study agent administration (Day 0) through 8 weeks following administration of the last dose of study agent (4 weeks in subjects entering the continuation protocol) are recorded on the Adverse Event Case Report Form (AE eCRF). All data fields on the AE eCRD are completed.

Serious Adverse Events (SAEs) are recorded on the SAE Worksheet and sent to the Human Genome Science Drug Safety designee and the FDA within 24 hours of site personnel becoming aware of the SAE. The SAE Worksheet is completed as much as possible but should not be held until all information is available. Additional information, follow-up information, and corrections are provided on subsequent updates of the SAE Worksheet that are clearly identified as follow-up (#1, #2, etc) reports. The SAE Worksheets are sent to the sponsor using the Drug Safety contact information and process as provided in the Study Procedures Manual.

Drug Safety personnel are available to answer questions and assist site personnel in documenting SAEs and completing the SAE worksheet.

All SAEs that are considered unexpected and related to the study agent are reported by the sponsor or designee as expedited reports to the appropriate regulatory authorities and to all participating investigators. In addition, the sponsor or designee follows all applicable local and national regulatory requirements regarding safety reporting. Each investigator also complies with the applicable regulatory requirements related to the reporting of SAEs to the Institutional Review Board (IRB)/Independent Ethics Committee (IEC) responsible for reviewing the study at their site, as well as the regulatory authority(ies) (if applicable).

### Other Events Requiring Rapid Reporting (Protocol Specified Events)

Protocol Specified Events (PSEs) are additional events that are reported to the Drug Safety designee in an expedited manner. Severe and life-threatening (Grade 3 and Grade 4) infections are PSEs for this protocol. PSEs may or may not be AEs or SAEs as defined in this protocol. PSEs are considered SAEs if they meet 1 or more of the criteria for an SAE. PSEs are recorded on SAE Worksheets and sent to the Drug Safety designee within 24 hours of site personnel becoming aware of the event.

### Pregnancies

Pregnant women are discontinued treatment with belimumab and receive therapy according to the investigator's judgment.

A pregnancy is reported to the Drug Safety designee as soon as the site becomes aware of the pregnancy. All pregnancies are reported up to 8 weeks following the last dose of study agent. The Drug Safety designee sendsi send an acknowledgement memorandum to the principal investigator along with a Pregnancy Assessment Form. A follow-up Pregnancy Assessment Form is sent to the site every 3 months. Pregnancy Assessment Forms are completed by the investigator until delivery, elective termination of the pregnancy, or miscarriage. The investigator is responsible for following the subject's pregnancy to final outcome.

Pregnancies are not considered adverse events. Complications or medical problems associated with a pregnancy are considered AEs and may be SAEs. Complications or medical problems are reported as AEs/SAEs if they occur during the study follow-up period (8 weeks after the last dose of study agent).

### Data Monitoring Board

A formal data safety monitoring board are constituted. After follow-up of the first 5 and 10 patients for 3 and 6 months, respectively, interim analysis data of clinical outcomes are reviewed by the data safety monitoring board and investigators.

### Example 6

### Data Review and Database Management

### Site Monitoring

As this is an investigator initiated project, no study monitoring is performed.

### Data Collection

Designated investigator staff must enter the information required by the protocol onto the CRFs.

### Database Management and Quality Control

Data from the CRFs is entered into a study database.

### Example 7

### Data Analysis

The following populations are determined for the statistical analyses: Intent to Treat (ITT) Population: All patients who are enrolled into the study and received at least one dose of belimumab Per Protocol (PP) Population; all patients who are enrolled into the study and completed the full 12 month course of treatment with belimumab All data will be summarized using the ITT Population.

The Safety Population are to consist of all patients in the ITT population who had at least one post-baseline safety assessment. Of note, the statement that a patient had no adverse events also constitutes a safety assessment.

### Patient Demographics/Other Baseline Characteristics

Demographic and background information are summarized by means of frequency distributions (for categorical variables) and descriptive statistics of mean, standard deviation, minimum, median, and maximum (for continuous variables). Background information includes prior medication, past/current medical conditions, and transplant history.

### Treatments (Study Drug, Concomitant Therapies, Compliance)

The doses of study medication (belimumab) received are listed for each patient by visit, and numbers and percentages presented in frequency tables.

Concomitant medication is coded according to the ATC (Anatomical, Therapeutic, and Chemical) classification system, and are tabulated descriptively.

Having described the embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method for reducing the level of alloantibodies in a subject awaiting kidney transplantation, comprising the step of administering to said subject a therapeutically effective amount of Belimumab, thereby reducing the level of alloantibodies in said subject awaiting kidney transplantation.

2. The method of claim 1, whereby said subject awaiting kidney transplantation is a subject afflicted with end-stage renal disease.

3. The method of claim 1, whereby said subject awaiting kidney transplantation is a subject having a Panel Reactive Antibody (PRA) measure from 5% to 99%.

4. The method of claim 1, whereby said reducing the level of alloantibodies is reducing the level of anti-HLA alloantibodies.

5. The method of claim 1, whereby said subject awaiting kidney transplantation is a subject who underwent a prior organ transplantation or a blood transfusion, or experienced a pregnancy, or any combination thereof.

6. A method for reducing the level of alloantibodies in a subject in need thereof, comprising the step of administering to said subject a therapeutically effective amount of a composition comprising an antibody that specifically binds a B-lymphocyte stimulator protein (BLyS), thereby reducing the level of alloantibodies in said subject in need thereof.

7. The method of claim 6, whereby said antibody that specifically binds a BLyS is a monoclonal antibody, a chimeric antibody, a humanized antibody, or a single chain variable fragment antibody.

8. The method of claim 6, whereby said antibody that specifically binds a BLyS is an IgG1λ antibody.

9. The method of claim 6, whereby said antibody that specifically binds a BLyS, further differentially binds a soluble BLyS and not a membrane bound BLyS.

10. The method of claim 6, whereby said antibody that specifically binds a BLyS is an antibody that specifically binds a human BLyS.

11. The method of claim 6, whereby said composition comprising an antibody that specifically binds a BLyS is Belimumab.

12. The method of claim 6, whereby said subject in need thereof is a subject awaiting an organ transplantation.

13. The method of claim 6, whereby said subject in need thereof is a subject awaiting kidney transplantation.

14. The method of claim 6, whereby said subject in need thereof is a subject afflicted with an end-stage renal disease.

15. The method of claim 6, whereby said subject in need thereof is a subject having a Panel Reactive Antibody (PRA) measure from 5% to 99%.

16. The method of claim 6, whereby said alloantibodies are anti-HLA alloantibodies.

17. The method of claim 6, whereby said subject in need thereof is a subject who underwent a prior organ transplantation or a blood transfusion, or experienced a pregnancy, or any combination thereof.

18. A method for reducing the risk of an allograft rejection in an allograft recipient, comprising the step of administering to said recipient a therapeutically effective amount of a composition comprising an antibody that specifically binds a B-lymphocyte stimulator protein (BLyS), wherein administering is prior to an organ transplantation, thereby reducing the risk of an allograft rejection in said allograft recipient.

19. The method of claim 18, whereby said reducing the risk of a graft rejection is seroconverting a positive cross match recipient to a negative cross match recipient.

20. The method of claim 18, whereby said antibody that specifically binds a BLyS is a monoclonal antibody, a chimeric antibody, a humanized antibody, or a single chain variable fragment antibody.

21. The method of claim 18, whereby said antibody that specifically binds a BLyS is a is an IgG1λ antibody

22. The method of claim 18, whereby said antibody that specifically binds a BLyS, further differentially binds a soluble BLyS and not a membrane bound BLyS.

23. The method of claim 18, whereby said antibody that specifically binds a BLyS is an antibody that specifically binds a human BLyS.

24. The method of claim 18, whereby said composition comprising an antibody that specifically binds a BLyS is Belimumab.

25. The method of claim 18, whereby said allograft is a kidney allograft.

26. The method of claim 18, whereby said rejection is hyperacute rejection.

27. The method of claim 18, whereby said reducing the risk of allograft rejection in said recipient is reducing the level of alloantibodies in said recipient.

28. The method of claim 27, whereby said alloantibodies are anti-HLA alloantibodies.

29. The method of claim 18, whereby said recipient underwent a prior organ transplantation or a blood transfusion, or experienced a pregnancy, or any combination thereof.

30. A method for reducing the wait time for an organ transplant in a patient in need thereof, comprising the step of administering to said patient a therapeutically effective amount of a composition comprising an antibody that specifically binds a B-lymphocyte stimulator protein (BLyS), thereby reducing the wait time for an organ transplant in said patient.

31. The method of claim 30, whereby said reducing the wait time for an organ transplant is reducing a Panel Reactive Antibody (PRA) measure in said patient.

32. The method of claim 30, whereby said antibody that specifically binds a BLyS is a monoclonal antibody, a chimeric antibody, a humanized antibody, or a single chain variable fragment antibody.

33. The method of claim 30, whereby said antibody that specifically binds a BLyS is an IgG1λ antibody.

34. The method of claim 30, whereby said antibody that specifically binds a BLyS, further differentially binds a soluble BLyS and not a membrane bound BLyS.

35. The method of claim 30, whereby said antibody that specifically binds a BLyS is an antibody that specifically binds a human BLyS.

36. The method of claim 30, whereby said composition comprising an antibody that specifically binds a BLyS is Belimumab.

37. The method of claim 30, whereby said organ is kidney.

38. The method of claim 30, whereby said reducing the wait time for an organ transplant in a patient in need thereof is reducing the level of alloantibodies in said patient.

39. The method of claim 38, whereby said alloantibodies are anti-HLA alloantibodies.

40. The method of claim 30, whereby said patient underwent a prior organ transplantation or a blood transfusion, or experienced a pregnancy, or any combination thereof.

41. A method of preventing the development of post-transplantation hyperacute rejection in a subject awaiting kidney transplantation, comprising the step of administering to said subject a therapeutically effective amount of a composition comprising an antibody that specifically binds a B-lymphocyte stimulator protein (BLyS), thereby preventing the development of post-transplantation hyperacute rejection in said subject awaiting kidney transplantation.

42. The method of claim 41, whereby said antibody that specifically binds a BLyS is a monoclonal antibody, a chimeric antibody, a humanized antibody, or a single chain variable fragment antibody.

43. The method of claim 41, whereby said antibody that specifically binds a BLyS is an IgG1λ antibody.

44. The method of claim 41, whereby said antibody that specifically binds a BLyS, further differentially binds a soluble BLyS and not a membrane bound BLyS.

45. The method of claim 41, whereby said antibody that specifically binds a BLyS is an antibody that specifically binds a human BLyS.

46. The method of claim 41, whereby said composition comprising an antibody that specifically binds a BLyS is Belimumab.
